(19) United States
(12) Patent Application Publication
(10) Pub. No.: US 2024/0245288 A1
(43) Pub. Date: Jul. 25, 2024

(54) SYSTEMS AND METHODS FOR LASER-BASED MEDICAL DEVICE ILLUMINATION

(71) Applicant: Noah Medical Corporation, San Carlos, CA (US)

(72) Inventor: John Thomas SHEN, San Jose, CA (US)

(21) Appl. No.: 18/605,303

(22) Filed: Mar. 14, 2024

Related U.S. Application Data
(63) Continuation of application No. PCT/US2022/043648, filed on Sep. 15, 2022.
(60) Provisional application No. 63/252,410, filed on Oct. 5, 2021.

(51) Int. Cl. A61B 1/06; A61B 1/00; A61B 1/005; A61B 1/05; A61B 1/07

(52) U.S. Cl. CPC A61B 1/0669; A61B 1/00117; A61B 1/005; A61B 1/05; A61B 1/063; A61B 1/0638; A61B 1/07

(57) ABSTRACT

A medical device is provided. The medical device comprises an articulating elongate member comprising a distal end, an imaging sensor is located at the distal end of the articulating elongate member; one or more laser light sources located at a proximal structure coupled to the articulating elongate member, where the one or more laser light sources generate light transmitted through one or more optic fibers; and one or more optical elements located at the distal end of the articulating elongate member and are configured to shape the light received via the one or more optic fibers.

### REFERENCE

This application is a Continuation application of International Application No. PCT/US2022/043648, filed Sep. 15, 2022, which claims priority to U.S. Provisional Patent Application No. 63/252,410, filed on Oct. 5, 2021, which is entirely incorporated herein by reference.

### BACKGROUND

Robotics technology has advantages that can be incorporated into endoscopes for a variety of applications (e.g., bronchoscope). For example, by exploiting soft deformable structures that are capable of moving effectively through a complex environment like inside the main bronchi, one can significantly reduce pain and patient discomfort. During endoscopy, an endoscopic system equipped with sensors such as electromagnetic (EM) three-dimensional (3D) sensors may register itself to the CT image or the patient anatomy. The EM information along with a direct visualization system (e.g., camera) may allow a physician to manipulate the endoscope (e.g., bronchoscope) to the site of the lesion and/or identify medical conditions based on the direct vision. However, the vision capability of such robotic endoscopes may still be challenging due to the insufficient illumination and the complexity and dynamic environment inside the patient body. In particular, an endoluminal robotic system may be limited by the size and heat dissipation constraints of the device.

### SUMMARY

Recognized herein is a need for enhanced vision capability with improved illumination. The present disclosure provides methods and apparatuses allowing for improved illumination for an endoscopic system. The improved illumination beneficially enhances the direct vision (e.g., camera) of the endoscopic system. Additionally, the present disclosure provides low-cost, single-use articulatable endoscope for diagnosis and treatment in various applications such as bronchoscopy, urology, gynecology, arthroscopy, orthopedics, ENT, gastro-intestine endoscopy, neurosurgery, and various others. It should be noted that the provided endoscope systems and/or the illumination methods can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others.

In an aspect, a medical device is provided. The medical device comprises: an articulating elongate member comprising a distal end, where an imaging sensor is located at the distal end of the articulating elongate member; one or more laser light sources located at a proximal component coupled to the articulating elongate member, where the one or more laser light sources generate light transmitted through one or more optic fibers; and one or more optical elements located at the distal end of the articulating elongate member, where the one or more optical elements receive the light via the one or more optic fibers and are configured to adjust a distribution of the light for illuminating a target scene based at least in part on a property of the imaging sensor or a property of the target scene.

In some embodiments, the medical device further comprises a coupler to mix the light from the one or more laser light sources into white light. In some embodiments, the articulating elongate member is releasably coupled to the proximal component, and where the articulating elongate member is disposable. In some embodiments, the medical device further comprises a laser speckle reducer located at the proximal component.

In some embodiments, the one or more laser light sources comprise at least a red laser diode, a green laser diode and a blue laser diode. In some cases, the one or more optical elements are selected based at least in part on the operating wavelength of the light received via a respective optic fiber.

In some embodiments, the property of the imaging sensor comprises at least one of field of view; sensitivity, acquisition parameter and exposure. In some cases, the distribution of the light is adjusted to match the field of view of the imaging sensor.

In some embodiments, the imaging sensor is embedded in the distal end of the articulating elongate member. In some embodiments, the one or more optical elements comprise at least one diffractive optic element. In some cases, the one or more optical elements further comprise an optical element for generating structured light that is used for creating a depth map.

In another aspect, an illumination system is provided for a medical device. The illumination system comprises: one or more laser light sources located at a proximal component of the medical device, where the proximal component is coupled to an articulating elongate member of the medical device; one or more optic fibers for transmitting light generated by the one or more laser light to a distal end of the articulating elongate member; and one or more optical elements located at the distal end of the articulating elongate member, where the one or more optical elements receive the light via the one or more optic fibers and are configured to adjust a distribution of the light for illuminating a target scene based at least in part on a property of an imaging sensor or a property of the target scene.

In some embodiments, the illumination system further comprises a coupler to mix the light from the one or more laser light sources into white light. In some embodiments, the articulating elongate member is releasably coupled to the proximal component, and where the articulating elongate member is disposable. In some embodiments, the illumination system further comprises a laser speckle reducer located at the proximal component.

In some embodiments, the one or more laser light sources comprise at least a red laser diode, a green laser diode and a blue laser diode. In some cases, the one or more optical elements are selected based at least in part on the operating wavelength of the light received via a respective optic fiber. In some embodiments, the property of the imaging sensor comprises at least one of field of view, sensitivity, acquisition parameter and exposure. In some cases, the distribution of the light is adjusted to match the field of view of the imaging sensor.

In some embodiments, the imaging sensor is embedded in the distal end of the articulating elongate member. In some embodiments, the one or more optical elements comprise at least one diffractive optic element. In some cases, the one or more optical elements further comprise an optical element for generating structured light that is used for creating a depth map.

It should be noted that the provided illumination components, methods, endoscope components and various components of the device can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of an assembly of an endoscope system, in accordance with some embodiments of the present disclosure.

FIG. 2 shows an example of a robotic endoscope, in accordance with some embodiments of the invention.

FIG. 3 shows an example of an instrument driving mechanism providing a mechanical and electrical interface to a handle portion of a robotic endoscope, in accordance with some embodiments of the invention.

FIG. 4 shows an example of a medical instrument with an illumination system, in accordance with some embodiments of the present disclosure.

FIG. 5 shows an example of a configuration of a distal portion of the medical instrument, in accordance with some embodiments of the present disclosure.

FIG. 6 shows examples of different configurations or placements of an imaging device and one or more diffractive optical elements (DOEs) at the distal tip of the medical instrument, in accordance with some embodiments of the present disclosure.

FIG. 7 shows an example of configuration utilizing multiple fibers, in accordance with some embodiments of the present disclosure.

FIG. 8 shows an example of configuration utilizing multiple fibers with multiple DOEs, in accordance with some embodiments of the present disclosure.

FIG. 9 shows an example of configuration utilizing multiple fibers with multiple DOEs distributed across a distal tip, in accordance with some embodiments of the present disclosure.

FIG. 10 shows an example of controlling the illumination for different regions of a target scene.

FIG. 11 schematically shows an example of using a combination of different DOEs.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

While exemplary embodiments will be primarily directed at an endoscope, robotic bronchoscope, or flexible instrument, one of skill in the art will appreciate that this is not intended to be limiting, and the devices described herein may be used for other therapeutic or diagnostic procedures and in other anatomical regions of a patient's body such as a digestive system, including but not limited to the esophagus, liver, stomach, colon, urinary tract, or a respiratory system, including but not limited to the bronchus, the lung, and various others.

The embodiments disclosed herein can be combined in one or more of many ways to provide improved diagnosis and therapy to a patient. The disclosed embodiments can be combined with existing methods and apparatus to provide improved treatment and visualization, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

The methods and apparatus as described herein can be used to treat any tissue of the body and any organ and vessel of the body such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat.

Whenever the term “at least.” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than.” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example. In some cases, the one or more processors may be a programmable processor (e.g., a central processing unit (CPU), graphic processing unit (GPU), or a microcontroller), digital signal processors (DSPs), a field programmable gate array (FPGA) and/or one or more Advanced RISC Machine (ARM) processors. In some cases, the one or more processors may be operatively coupled to a non-transitory computer readable medium. The non-transitory computer readable medium can store logic, code, and/or program instructions executable by the one or more processors unit for performing one or more steps. The non-transitory computer readable medium can include one or more memory units (e.g., removable media or external storage such as an SD card or random access memory (RAM)). One or more methods or operations disclosed herein can be implemented in hardware components or combinations of hardware and software such as, for example, ASICs, special purpose computers, or general purpose computers.

As used herein, the terms distal and proximal may generally refer to locations referenced from the apparatus, and can be opposite of anatomical references. For example, a distal location of an endoscope or catheter may correspond to a proximal location of an elongate member of the patient, and a proximal location of the endoscope or catheter may correspond to a distal location of the elongate member of the patient.

An endoscope system as described herein, includes an elongate portion or elongate member such as a catheter. The terms “elongate member” and “catheter” are used interchangeably throughout the specification unless contexts suggest otherwise. The elongate member can be placed directly into the body lumen or a body cavity. In some embodiments, the system may further include a support apparatus such as a robotic manipulator (e.g., robotic arm) to drive, support, position or control the movements and/or operation of the elongate member. Alternatively or in addition to, the support apparatus may be a hand-held device or other control devices that may or may not include a robotic system. In some embodiments, the system may further include peripheral devices and subsystems such as imaging systems that would assist and/or facilitate the navigation of the elongate member to the target site in the body of a subject.

The endoscope system of the present disclosure may provide enhanced vision capability with improved illumination. In some embodiments, the sensing system may comprise at least direct vision (e.g., camera). The sensing system may also comprise positional sensing (e.g., EM sensor system, optical shape sensor, accelerometers, gyroscopic sensors), or other modalities such as ultrasound imaging.

The direct vision may be provided by an imaging device such as a camera. A camera may comprise imaging optics (e.g. lens elements) and image sensor (e.g. CMOS or CCD). The field of view of the imaging device may be illuminated by an illumination system described later herein (e.g. laser-based light source). The imaging device may be located at the distal tip of the catheter or elongate member of the endoscope. In some cases, the direct vision system may comprise an imaging device and an illumination device. In some embodiments, the imaging device may be a video camera. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor and circuitry to combine or serialize the data so that it can be transmitted in a minimum number of electrical conductors. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors.

In some cases, the endoscope system may implement a positional sensing system such as electromagnetic (EM) sensor, fiber optic sensors, and/or other sensors to register and display a medical implement together with preoperatively recorded surgical images thereby locating a distal portion of the endoscope with respect to a patient body or global reference frame. The position sensor may be a component of an EM sensor system including one or more conductive coils that may be subjected to an externally generated electromagnetic field. Each coil of EM sensor system used to implement positional sensor system then produces an induced electrical signal having characteristics that depend on the position and orientation of the coil relative to the externally generated electromagnetic field. In some cases, an EM sensor system used to implement the positional sensing system may be configured and positioned to measure at least three degrees of freedom e.g., three position coordinates X, Y, Z. Alternatively or in addition to, the EM sensor system may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

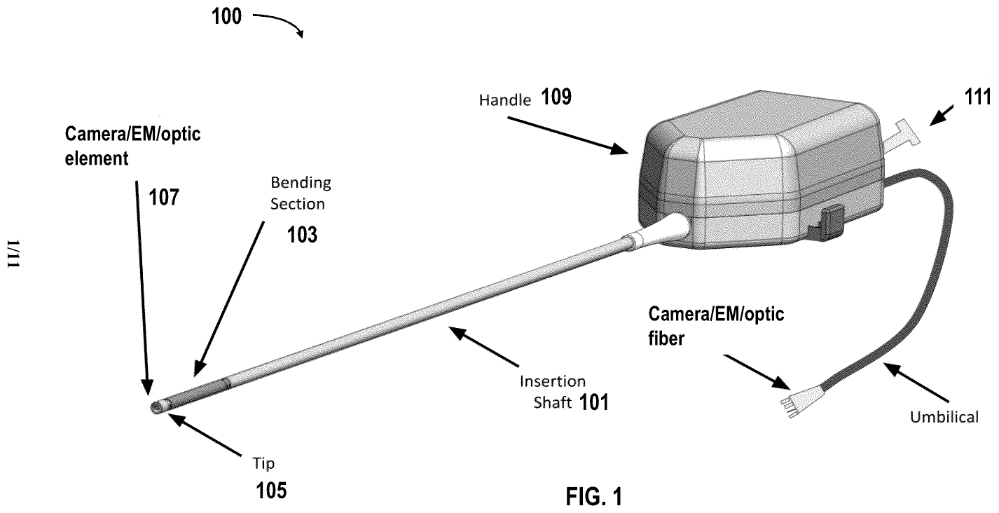

In an aspect of the invention, a flexible endoscope with improved performance at reduced cost is provided. FIG. 1 illustrates an example of a flexible endoscope 100, in accordance with some embodiments of the present disclosure. As shown in FIG. 1, the flexible endoscope 100 may comprise a handle portion 109 and a flexible elongate member to be inserted inside of a subject. In some embodiments, the flexible elongate member may comprise a shaft (e.g., insertion shaft 101), steerable tip (e.g., tip 105) and a steerable section (bending section 103). The endoscope 100 may also be referred to as steerable catheter assembly as described elsewhere herein. In some cases, the endoscope 100 may be a single-use robotic endoscope. In some cases, the entire catheter assembly may be disposable. In some cases, at least a portion of the catheter assembly may be disposable. In some cases, the entire endoscope may be released from an instrument driving mechanism and can be disposed of. In some embodiment, the endoscope may contain varying levels of stiffness along the shaft, as to improve functional operation.

The endoscope or steerable catheter assembly 100 may comprise a handle portion 109 that may include one or more components configured to process image data, provide power, or establish communication with other external devices. For instance, the handle portion may include a circuitry and communication elements that enables electrical communication between the steerable catheter assembly 100 and an instrument driving mechanism (not shown), and any other external system or devices. In another example, the handle portion 109 may comprise circuitry elements such as power sources for powering the electronics (e.g. camera, electromagnetic sensor) of the endoscope. In some cases, a light source assembly (including one or more laser sources) of the illumination system may be located at the handle portion. Alternatively, the light source assembly may be located at the instrument driving mechanism, the robotic support system or hand-held controller. Details about the illumination system are described later herein.

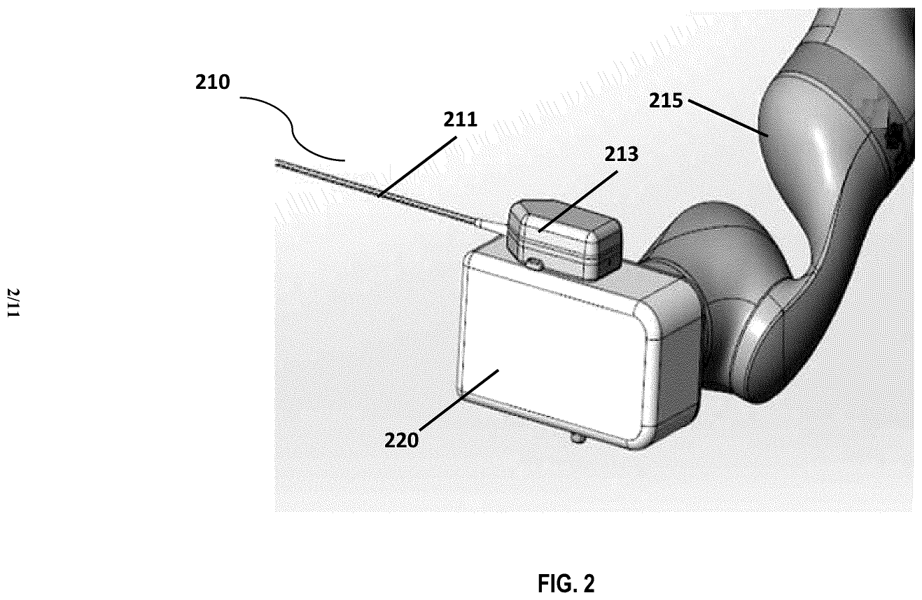

The one or more components located at the handle may be optimized such that expensive and complicated components may be allocated to the robotic support system, a hand-held controller or an instrument driving mechanism thereby reducing the cost and simplifying the design the disposable endoscope. In some cases, the handle portion may be in electrical communication with the instrument driving mechanism (e.g., FIG. 2, instrument driving mechanism 220) via an electrical interface (e.g., printed circuit board) so that image/video data and/or sensor data can be received by the communication module of the instrument driving mechanism and may be transmitted to other external devices/systems. In some cases, the electrical interface may comprise an optical interface such as connector interface for the illumination system (e.g., optic fiber connector).

In some cases, the electrical interface may establish electrical communication without cables or wires. For example, the interface may comprise pins soldered onto an electronics board such as a printed circuit board (PCB). For instance, receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

In some cases, the handle portion 109 may comprise one or more mechanical control modules such as lure 111 for interfacing the irrigation system/aspiration system. In some cases, the handle portion may include lever/knob for articulation control. Alternatively, the articulation control may be located at a separate controller attached to the handle portion via the instrument driving mechanism.

The endoscope may be attached to a robotic support system or a hand-held controller via the instrument driving mechanism. The instrument driving mechanism may be provided by any suitable controller device (e.g., hand-held controller) that may or may not include a robotic system. The instrument driving mechanism may provide mechanical and electrical interface to the steerable catheter assembly 100. The mechanical interface may allow the steerable catheter assembly 100 to be releasably coupled to the instrument driving mechanism. For instance, a handle portion of the steerable catheter assembly can be attached to the instrument driving mechanism via quick install/release means, such as magnets, spring-loaded levels and the like. In some cases, the steerable catheter assembly may be coupled to or released from the instrument driving mechanism manually without using a tool.

In the illustrated example, the distal tip of the catheter or endoscope shaft is configured to be articulated/bent in two or more degrees of freedom to provide a desired camera view or control the direction of the endoscope. As illustrated in the example, imaging device (e.g., camera), position sensors (e.g., electromagnetic sensor), and one or more optic elements (e.g., diffractive optic element) 107 is located at the tip of the catheter or endoscope shaft 105. For example, line of sight of the camera may be controlled by controlling the articulation of the bending section 103. In some instances, the angle of the camera may be adjustable such that the line of sight can be adjusted without or in addition to articulating the distal tip of the catheter or endoscope shaft. For example, the camera may be oriented at an angle (e.g., tilt) with respect to the axial direction of the tip of the endoscope with aid of an optimal component.

The distal tip 105 may be a rigid component that allow for positioning sensors such as electromagnetic (EM) sensors, imaging devices (e.g., camera) and illuminating component elements (e.g., diffractive optic element) or other electronic components (e.g., LED light source) being embedded at the distal tip.

In real-time EM tracking, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. For example, the EM field generator may be positioned close to the patient torso during procedure to locate the EM sensor position in 3D space or may locate the EM sensor position and orientation in 5D or 6D space. This may provide a visual guide to an operator when driving the bronchoscope towards the target site. Details about the tip design and the plurality of components embedded at the tip are described later herein.

The endoscope may have a unique design in the shaft component. In some cases, the insertion shaft of the endoscope may consist of a single tube that incorporates a series of cuts (e.g., reliefs, slits, etc.) along its length to allow for improved flexibility as well as a desirable stiffness.

The bending section 103 may be designed to allow for bending in two or more degrees of freedom (e.g., articulation). A greater bending degree such as 180 and 270 degrees (or other articulation parameters for clinical indications) can be achieved by the unique structure of the bending section. In some cases, the bending section may be fabricated separately as a modular component and assembled to the insertion shaft. In some cases, the bending section may further incorporate minimalist features thereby reducing cost and increasing reliability. For example, the bending section may incorporate a cut pattern that beneficially allows for a greater degree of tube deflection to achieve a desired tip displacement relative to the insertion shaft.

FIG. 2 shows an example of a robotic endoscope system supported by a robotic support system. In some cases, the handle portion may be in electrical communication with the instrument driving mechanism (e.g., instrument driving mechanism 220) via an electrical interface (e.g., printed circuit board) so that image/video data and/or sensor data can be received by the communication module of the instrument driving mechanism and may be transmitted to other external devices/systems. In some embodiments, the electrical interface may also comprise optic interface or connector for illumination fibers of the illumination system. In some cases, the electrical interface may establish electrical communication without cables or wires. For example, the interface may comprise pins soldered onto an electronics board such as a printed circuit board (PCB). For instance, receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

As shown in FIG. 2, a robotic endoscope 220 may comprise a handle portion 213 and a flexible elongate member 211. In some embodiments, the flexible elongate member 211 may comprise a shaft, steerable tip and a steerable (bending) section. The robotic endoscope 220 can be the same as the steerable catheter assembly as described in FIG. 1. The robotic endoscope may be a single-use robotic endoscope. In some cases, only the catheter may be disposable. In some cases, at least a portion of the catheter may be disposable. In some cases, the entire robotic endoscope may be released from the instrument driving mechanism and can be disposed of. The endoscope may contain varying levels of stiffness along its shaft, as to improve functional operation.

The robotic endoscope can be releasably coupled to an instrument driving mechanism 220. The instrument driving mechanism 220 may be mounted to the arm 215 of the robotic support system or to any actuated support system as described elsewhere herein. The instrument driving mechanism may provide mechanical and electrical interface to the robotic endoscope 220. The mechanical interface may allow the robotic endoscope 220 to be releasably coupled to the instrument driving mechanism. For instance, the handle portion of the robotic endoscope can be attached to the instrument driving mechanism via quick install/release means, such as magnets and spring-loaded levels. In some cases, the robotic endoscope may be coupled or released from the instrument driving mechanism manually without using a tool.

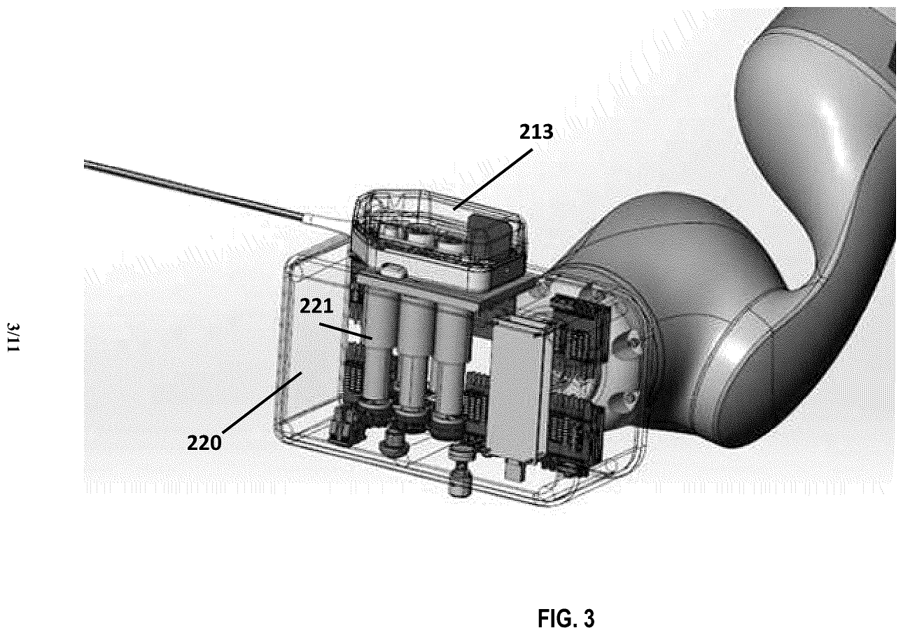

FIG. 3 shows an example of an instrument driving mechanism 220 providing mechanical interface to the handle portion 213 of the robotic endoscope. As shown in the example, the instrument driving mechanism 220 may comprise a set of motors 221 that are actuated to rotationally drive a set of pull wires of the catheter. The handle portion 213 of the catheter assembly may be mounted onto the instrument drive mechanism so that its pulley assemblies are driven by the set of motors. The number of pulleys may vary based on the pull wire configurations. In some cases, one, two, three, four, or more pull wires may be utilized for articulating the catheter.

The handle portion may be designed allowing the robotic endoscope to be disposable at reduced cost. For instance, classic manual and robotic endoscope may have a cable in the proximal end of the endoscope handle. The cable often includes camera video cable, and other sensors fibers or cables such as electromagnetic (EM) sensors, or shape sensing fibers. Such complex cable can be expensive adding to the cost of the endoscope. The provided robotic endoscope may have an optimized design such that simplified structures and components can be employed while preserving the mechanical and electrical functionalities.

In some case, the handle portion may be housing or comprise components configured to process image data, provide power, or establish communication with other external devices. In some cases, the communication may be wireless communication. For example, the wireless communications may include Wi-Fi, radio communications, Bluetooth, IR communications, or other types of direct communications. Such wireless communication capability may allow the robotic bronchoscope function in a plug-and-play fashion and can be conveniently disposed after single use. In some cases, the handle portion may comprise circuitry elements such as power sources for powering the electronics (e.g. camera and LED light source) disposed within the robotic endoscope or catheter.

The handle portion may be designed in conjunction with the catheter such that cables can be reduced. For instance, the catheter portion may employ a design having working channel allowing instruments to pass through the robotic endoscope, low cost electronics such as a chip-on-tip camera, optics as part of the illumination system, and EM sensors located at optimal locations in accordance with the mechanical structure of the catheter. This may allow for a simplified design of the handle portion. For example, the handle portion may include a proximal board where the camera cable, optic fiber, and EM sensor cable terminate while the proximal board connects to the interface of the handle portion and establishes the electrical connections to the instrument driving mechanism. As described above, the instrument driving mechanism is attached to the robot arm (robotic support system) and provides a mechanical and electrical interface to the handle portion. This may advantageously improve the assembly and implementation efficiency as well as simplify the manufacturing process and cost. In some cases, the handle portion along with the catheter may be disposed of after a single use.

The catheter of the endoscope may include a lumen sized to receive a lumen or working channel to receive an instrument. Various instruments can be inserted through the lumen such as biopsy needle, graspers, scissors, baskets, snares, curette, laser fibers, stitching tools, balloons, morcellators, various implant or stent delivery devices, and the like.

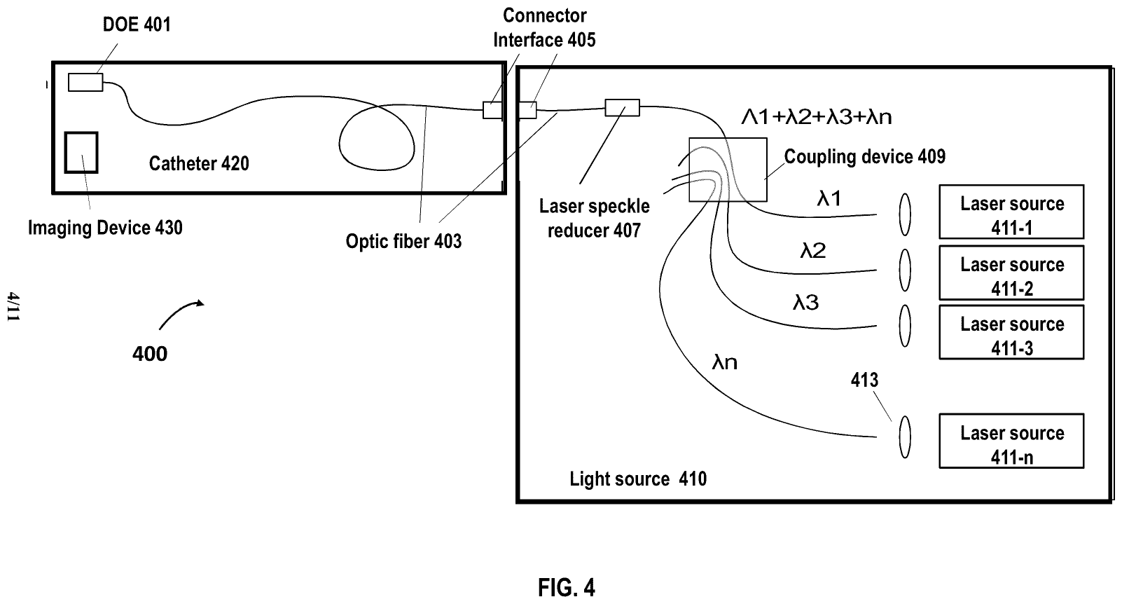

The imaging device, the illumination components (e.g., diffractive optical element (DOE)), EM sensor may be integrated to the distal tip of the catheter. For example, the distal portion of the catheter may comprise suitable structures matching at least a dimension of the above components. In some cases, the distal tip may have a dimension so that the one or more electronic components or optics can be embedded into the distal tip. For instance, the imaging device may be embedded into a cavity at the distal tip. The cavity may be integrally formed with the distal portion and may have a dimension matching a length/width of the camera such that the camera may not move relative to the distal tip. FIGS. 4-9 show various examples of the distal portion of the catheter with integrated imaging device and the illumination device.

In some cases, the power to the camera may be provided by a wired cable. In some cases, the cable wire may be in wire bundle providing power to the camera as well as other circuitry at the distal tip of the catheter. The camera may be supplied with power from a power source disposed in the handle portion of the catheter via wires, copper wires, or via any other suitable means running through the length of the hybrid probe. In some cases, real-time images or video of the tissue or organ may be transmitted to external user interface or display wirelessly. The wireless communication may be WiFi, Bluetooth, RF communication or other forms of communication. In some cases, images or videos captured by the camera may be broadcasted to a plurality of devices or systems. In some cases, image and/or video data from the camera may be transmitted down the length of the catheter to the processors situated in the handle portion via wires, copper wires, or via any other suitable means. The image or video data may be transmitted via the wireless communication component in the handle portion to an external device/system.

### Illumination System

In an aspect of the present disclosure, an improved illumination system is provided for a medical instrument. Compared with existing illumination method such as phosphor-coated blue LEDs, the illumination system herein may utilize laser diodes (LD) (e.g., red, green, and blue (RGB)) to generate a white-light source. In some cases, the white-light source may have a high color rendering index (CRI) producing a more accurate color rendering of the objects around it. This can be critical for diagnosis based on the color of tissues shown in the imaging. In some cases, the light sources may comprise additional visible wavelengths or selected wavelength to further improve the CRI performance. For example, by selecting a red LD at a longer wavelength may improve the color rendering index. In some cases, the light sources may comprise ultraviolet (UV) or infrared (IR) wavelengths for additional imaging modalities such as narrow-band imaging or fluorescence imaging.

Additionally, compared to illumination devices using phosphor or light conversion (which does not convert light 100% efficiently resulting in some of the light energy being converted into heat), the provided illumination system has higher efficiency without using light conversion thereby reducing the heat production. The laser light source of the illumination system allows the beam easily focused into a single small fiber with improved coupling efficiencies. This may further avoid excess heating at the coupler.

FIG. 4 shows an example of a medical instrument 400 with an illumination system. The illumination system may comprise a light source assembly 410 comprising a plurality of light sources. The plurality of light sources may comprise one or more laser light sources 411-1, 411-2, 411-3, 411-n. In some embodiments, the laser light sources may be centered at wavelengths of interest such as Red, Green, Blue, IR and/or UV. The use of laser light sources beneficially allows most of light to be delivered at fairly high efficiency. Multiple lasers of differing wavelengths can be easily coupled into the same optic fiber, allowing for improved color rendering for white light. The flexibility of coupling additional light sources or different combination of light sources also allows for advanced imaging applications such as fluorescence imaging or narrow band imaging. Details about the light sources are described later herein.

The medical instrument may be an endoscope as described elsewhere herein. In some embodiment, the light source assembly 410 may be located at a handle portion, an instrument driving mechanism, a robotic support system, or a hand-held controller. In some cases, the light source assembly may be located on a component that is releasably coupled to the catheter 420. For example, one or more laser diodes may be placed in the handle of the elongate medical instrument or the instrument driving mechanism. Additionally or alternatively, the light source assembly may be included in the robotic support system or a console remote from the medical instrument.

The light source assembly 410 may be optically coupled to the optics (e.g., optic fiber, diffractive optical element (DOE) 401) that located at the catheter via a connector interface 405. The connector interface 405 may include, for example, a coupler to connect the optic fiber 403. The connector interface 405 can be placed at various locations in the electric signal/illumination chain depending on a specific configuration of the system. For example, the connector interface 405 may be part of the electric interface between a disposable portion and reusable portion. In some cases, the connector interface 405 may located at the interface (e.g., electric and/or mechanical interface) between the handle and the instrument driving mechanism as described elsewhere herein. Alternatively, the connector interface may not be at the above electric interface. For instance, the connector interface may be located between the catheter and the handle portion, or at an interface between the instrument driving mechanism and the robotic arm support. Decoupling the light source assembly from the catheter may beneficially allow the light source assembly to be reused whereas the components at the catheter are disposable.

In some cases, one or more of the plurality of light sources may be coupled into a single optic fiber 403 (e.g., single mode or multimode) of the illumination system. The illumination system may comprise one or more optic fibers each is coupled to at least one light source. The one or more optic fibers 403 may run through the catheter 420 to the distal tip. Using fiber for transmission of the illumination light may drastically reduce the space needed for visualization components within the tube/lumen of the catheter or an endoluminal robotic device.

In some embodiments, the illumination system may comprise an optical component such as a diffractive optical element (DOE) 401 that directs the energy from the output end of the optic fiber 403 into a spatial distribution that may be desirable for the image sensor(s) that perform image acquisition by sensing photons. The DOE may shape the illumination light to better match the field of view of the imaging system. The transmission through the exit of the fiber and the optical element can have high efficiency (e.g., at least 90%), resulting in reduced heating compared to using imaging fiber bundles (which requires higher power) or using LEDs as light sources at the distal tip. In some cases, in addition to shaping the illumination light for the imaging sensor (e.g., camera) located at the distal tip, an additional DOE may be used to provide structured light to the scene to enhance depth map accuracy. Details about the DOE are described later herein.

In some cases, one or more optical fiber elements may be optically coupled to the set of light sources at an input end (i.e., end receiving light from a light source), and coupled to the DOE at an output end. In some cases, the illumination system may comprise a coupling device 409 at the light source assembly 410 for mixing the RGB lights into a white light or for mixing multiple wavelengths generated by multiple light sources into a desired illumination light that is appropriate for the imaging sensor.

In some cases, the size or dimension of the one or more optical fibers 403 may be selected such that multiple fibers may fit the dimension of the catheter. The flexibility of adding more optic fibers may enhance the total illumination power, provide for simpler optical design for multiple wavelengths, or a combination of both.

The one or more optical fibers may be single mode fiber or multimode fibers. In some cases, the optical fiber 403 may be a single mode fiber. For example, the optical fiber 403 may have an outer diameter (OD) that is about 0.1 mm, about 0.15 mm, about 0.2 mm, about 0.25 mm, about 0.3 mm, about 0.35 mm, about 0.4 mm, about 0.45 mm, about 0.5 mm, about 0.6 mm, about 0.7 mm, about 0.8 mm, about 0.9 mm, about 1 mm, have any OD value between, or have an OD that falls within any range having endpoints therein. The optic fiber may propagate the laser light to the endoscope tip (e.g., via total internal reflection (TIR)). In some cases, the optical fiber 403 may have a polymer coating that protects the fiber and may enable it to withstand more rugged conditions than certain delicate (e.g., borosilicate) fibers. The optical fiber 403 may emit the light at the tip directly into a DOE 401. One or more single mode fibers may run through the catheter and the overall size of the multiple single mode optic fibers may still be much smaller than an imaging fiber bundle or LED wires.

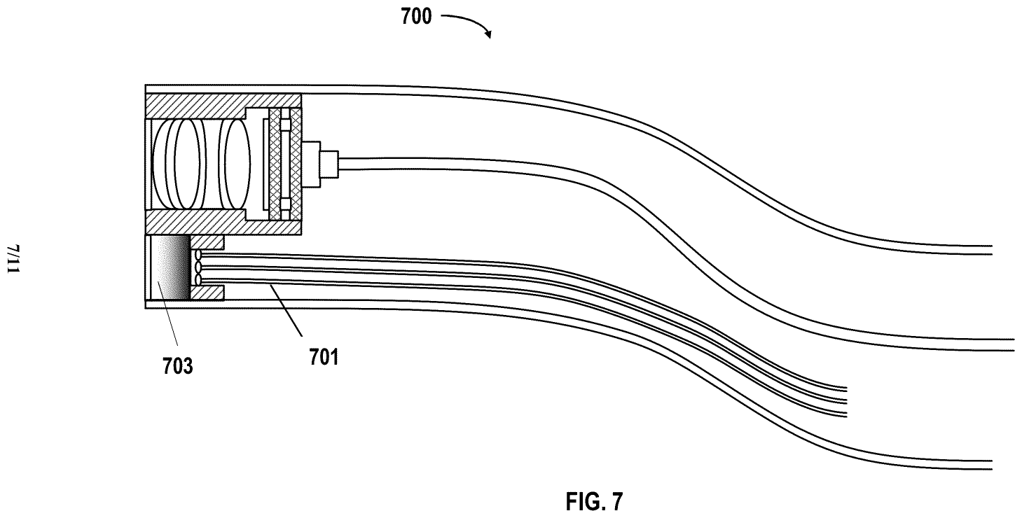

Employing multiple single mode optic fibers may allow for selecting optical elements for different light sources (e.g., example as shown in FIG. 7). For example, different optical elements may be selected corresponding to the properties of the light coming from each fiber where the light may be different. For example, different optic elements may be selected for LED light, laser beam, different wavelengths (e.g., UV, IR, etc.), different desired diffusion pattern, and the like. In some cases, different DOEs may be selected for multiple optic fibers and each DOE may be different depending on the configuration. For instance, the DOE may be selected to have an operating wavelength that matches to the dominant wavelength of light that is coupled to it by its fiber. For example, when the light wavelengths are different, the plurality of DOEs corresponding to the plurality of lights may be different by aligning the operating wavelength of the DOE to the corresponding laser wavelength.

In some embodiments, the light source assembly may comprise an optical coupler 413 configured to couple the light from the light source 411-1, 411-2, 411-3, . . . 411-n into the corresponding optical fiber. The light of the different wavelengths may be mixed into white light by a coupling device 409.

In some embodiments, the optical fiber 403 may be a multimode fiber. The optical fiber 403 may have an outer diameter (OD) that is about 0.1 mm, about 0.15 mm, about 0.2 mm, about 0.25 mm, about 0.3 mm, about 0.35 mm, about 0.4 mm, about 0.45 mm, about 0.5 mm, about 0.6 mm, about 0.7 mm, about 0.8 mm, about 0.9 mm, about 1 mm, have any OD value between, or have an OD that falls within any range having endpoints therein.

The illumination system may comprise a plurality of light sources 411-1, 411-2, 411-3, . . . 411-n. The plurality of light sources 411-1, 411-2, 411-3, . . . 411-n may comprise laser diodes having any suitable operating wavelength, such as for example, centered at red, green and blue. In some embodiments, the plurality of light sources may comprise red, green, and blue (RGB) laser diodes (LD) to generate a white-light source. The transmission capacity of individual Red. Green, and Blue LDs can be high compared to LEDs. This may beneficially reduce heat production. For example, a laser diode may generate a fraction (e.g., about one quarter) of the waste heat in the endoscope handle portion that may otherwise be generated with a white light LED but may have a comparable white light output at the distal tip of the endoscope. Additionally, the laser beam can be coupled into the fiber with high efficiency (e.g., greater than 90%) than a coupling of white light from a white light LED owning to the high transmission capacity of individual RGB laser beam and selecting the properties of the fiber optic as described above.

In some cases, in addition to laser diodes, other light source such as light-emitting diodes (LEDs), filaments, and/or other light emitting elements may be used to reduce the cost. For instance, the plurality of light sources may include a combination of LDs and LEDs to reduce the cost when higher power or efficiency is not needed. For example, a monochromatic LED (e.g., green, blue, red, etc.) may be used to replace the laser diode for the same wavelength.

In some cases, the plurality of light sources may comprise UV or IR wavelengths for additional imaging modalities such as narrow-band imaging or fluorescence imaging (e.g., using contrast agents), auto fluorescence imaging, multi-band imaging, and/or nonlinear imaging (e.g., light conversion at the tissue).

The illumination system may comprise RGB laser diodes to be mixed into white light without utilizing light conversion. However, laser light source may have speckle issue particularly in absence of light conversion (more coherent light than light source with light conversion) where the perceived image may be blurred, low contrast, and cause less safety. Speckle is the result of interference of light beams with the same frequency, but different phase and amplitude, resulting in a wave with random amplitude variations. The illumination system herein may provide an illumination light with good quality by reducing the speckle contrast using a laser speckle reducer.

In some embodiments, a laser speckle reducer 407 may be used in-line with the optical fibers at the light source to homogenize the light thereby reducing speckle contrast. The present disclosure provides efficient speckle-suppressed white light source or speckle-suppressed light source in any suitable wavelength range.

In some cases, a single laser speckle reducer 407 may be used for multiple laser sources 411-1, . . . , 411-n. The laser speckle reducer 407 may be coupled to the optical fiber and may be part of the light source. The laser beam generated by the laser diodes may pass the laser speckle reducer resulting in reduced speckle contrast.

The laser speckle reducer may be built into the light source assembly 410. For example, the laser speckle reducer 407 may be located at the handle portion. In some cases, the laser speckle reducer may be located prior to coupling into the optic fiber or located after coupling into the fiber but prior to exiting the light source assembly. In some cases, the laser speckle reducer may be located outside of the handle portion or located at the flexible elongated member such as built into the fiber chain close to the distal tip, or built into the distal tip.

In some embodiments, the laser speckle reducer 407 may be an optical downstream component that superimposes multiple speckle patterns at once so that on average no pattern is visible to the human eye. The laser speckle reducer 407 may utilize any suitable speckle reduction methods. For example, the laser speckle reducer 407 may employ techniques such as a laser array, polarization-rotation, dynamic deformable mirror, stationary multimode optical fiber, fast scanning micromirror, moving diffuser, rotating diffuser, encoding multiple holograms and/or other techniques.

In some cases, the laser speckle reducer 407 and the diffraction optical element (DOE) 401 may operate together to achieve a desired the speckle contrast reduction effect. In some cases, the laser speckle reducer 407 and the diffraction optical element (DOE) 401 may employ the same techniques or different techniques to achieve an overall speckle reduction effect. For example, the laser speckle reducer may employ a speckle reduction technique to reduce the speckle contrast whereas the DOE may be configured to diffuse or scatter the received light through the optic fiber to further widen the illumination angle or illumination cone to facilitate directing the light towards the desired illumination target.

In some cases, the diffractive optical element (DOE) 401 may be a passive element (e.g., diffractive optic element) coupled to a piezoelectric element to provide motion, or a rotating diffuser thereby shaping the illumination light to better match the field of view of the imaging system. Compared to conventional systems using phosphors, the DOE herein may withstand higher irradiance level, enabling an illumination system whose efficiency is mainly determined by that of the LDs used, due to the lack of any conversion effects. The DOE may employ any suitable techniques to achieve the desired spatial distribution. As described above, the DOE may direct the energy from the output end of the optic fiber 403 into a spatial distribution that may be desirable for the image sensor(s) in image acquisition. The DOE may function as a beam shaper that transform the incident laser beam (e.g., Gaussian intensity profile) into a desired intensity profile at the target scene. In some cases, the DOE may be controlled to adjust the output angle and intensity, modify the phase profiles of spatially coherent lasers or adjust the shape of the light distribution (e.g., circular, rectangular, or any other shape).

The DOE may beneficially provide flexibility for adjusting the spatial distribution (e.g., shape, location, intensity, intensity profile, etc.) of the illumination light. The spatial distribution may be adjusted based on, for example, requirement of the imaging device 430 (e.g., field of view, sensitivity range, acquisition parameters, exposure, etc.), application of the medical device, medical procedure, property of the target scene (e.g., reflectivity of the tissue inside patient body) and various others. For example, the DOE may be configured to diffuse or scatter the received light to widen the illumination angle or illumination cone to facilitate directing the light towards the desired illumination target. In some cases, the DOE may be adjusted or controlled (e.g., control motion of the piezoelectric element or rotation movement) to adjust the output angles to better match the field of view of the imaging device 430. In some cases, the DOE may provide angle independent (isotropic) emission over an entire angular range, so that the light is uniformly emitted in all directions. Alternatively or additionally, the DOE may provide uniform performance for a specific range at a defined distance from the distal end.

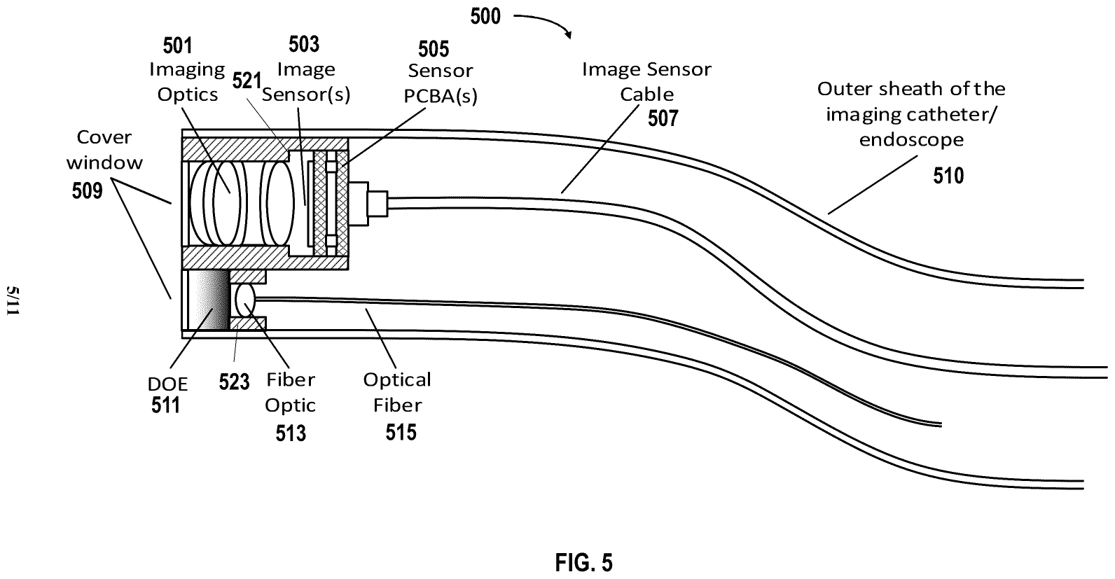

FIG. 5 shows an example of a configuration 500 of the distal portion of the system. In the illustrated example, the DOE 511 may be located at the distal tip comprising at least one optic fiber 515. The DOE may be located next to a cover window 509 and integral to the distal tip. For example, the distal tip may comprise a mechanical structure (e.g., cavity. recessive structure, etc.) receiving the DOE where glue may or may not be used. Alternatively, any other suitable fastening means may be utilized to secure the DOE. In some cases, the cover window 509 may be placed at the forwarding end face of the distal tip providing precise positioning of the DOE(s). In some cases, the cover window 509 may provide sufficient room for the glue of the DOE(s) and the cover 509 may be composed of transparent material matching the refractive index of the glue so that the illumination light may not be obstructed. In some cases, the cover window 509 may comprise a protection layer such as a thin layer of biocompatible glue applied to the front surface to provide protection while allowing light transmission.

The distal end of the optic fiber 515 may be terminated by a fiber optic 513 and fixed to the distal portion. In some cases, the fiber optic 513 may be configured to couple the focused mixed light into the center of the DOE 511 through the end face of the fiber optic at normal incidence.

The distal portion may also comprise an imaging device. The imaging device can be the same as the imaging device as described above. For example, the imaging device may comprise optical elements 501 and image sensor 503 for capturing image data. In some cases, power to the camera may be provided by a wired cable 507. In some cases, image and/or video data from the camera may be transmitted down the length of the probe 510 to the processors situated in the handle portion via wires 507, copper wires, or via any other suitable means. In some cases, image or video data may be transmitted via the wireless communication component in the handle portion to an external device/system. In some cases, real-time images or video of the tissue or organ may be transmitted to external user interface or display wirelessly. The wireless communication may be WiFi. Bluetooth. RF communication or other forms of communication.

The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board 505. The circuit board may be an imaging printed circuit board (PCB) 505. The PCB may comprise a plurality of electronic elements for processing the image signal. For instance, the circuit for a CCD sensor may comprise A/D converters and amplifiers to amplify and convert the analog signal provided by the CCD sensor and circuitry to combine or serialize the data so that it can be transmitted in a minimum number of electrical conductors. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors.

The imaging device may have parameters such as field of view, detection sensitivity and the like that may require a desirable illumination. As described above, the illumination system may be capable of adjusting the spatial distribution of the illumination based at least in part on the imaging device or image acquisition properties.

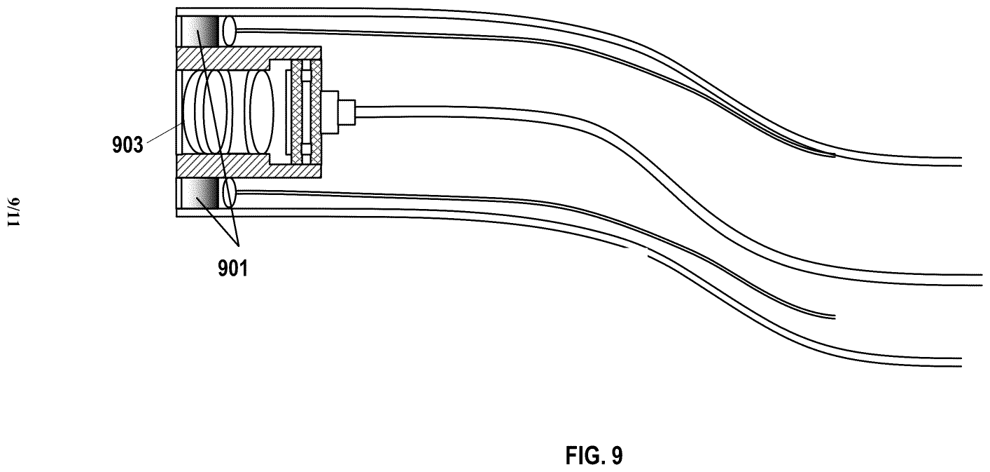

The imaging device, the illumination components (e.g., DOE), or EM sensor may be integrated to the distal tip. As shown in the example, the distal portion may comprise suitable structures matching at least a dimension of the above components. The distal tip may have a structure to receive the camera, illumination components (e.g., DOE) and/or the location sensor. For example, the camera may be embedded into a cavity 521 at the distal tip of the catheter. The cavity may be integrally formed with the distal portion of the cavity and may have a dimension matching a length/width of the camera such that the camera may not move relative to the catheter. In some cases, the distal portion may comprise a structure 523 having a dimension matching a dimension of the DOE 511. As shown in the example illustrated in FIG. 9, two cavities may be integrally formed with the distal portion to receive two (sets of) DOEs. Any number of DOEs may be included. The internal structure of the distal portion may be designed to fit any number of DOEs.

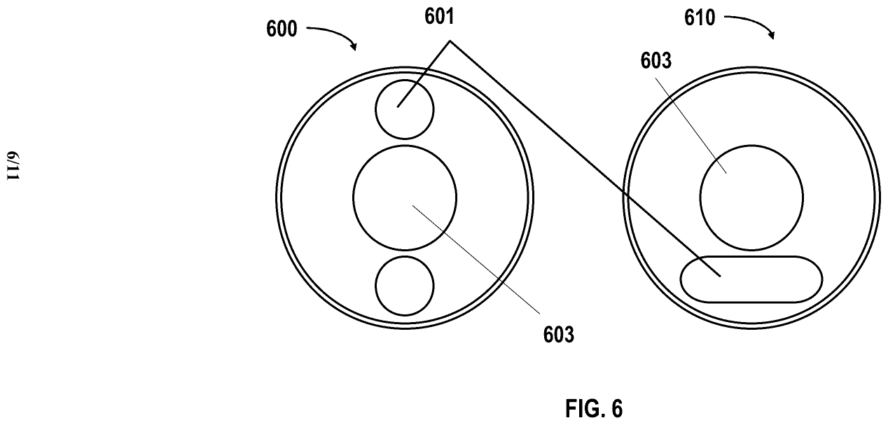

The imaging device and the DOE (or distal of the fiber optic) may have any suitable configurations. FIG. 6 shows examples of different configurations or placements of the imaging device and the DOE(s). One or more optic fibers/DOEs may be utilized by the system. The DOEs may be placed around the imaging device 603 such as at various locations 601. In one example 600, the DOEs may be displaced at locations spatially distributed surrounding the imaging device. In another example 610, the DOEs may be placed non-uniformly such as concentrated on one side of the imaging device. Based on the DOE(s) placement, the corresponding spatial response may be different. For example, the DOE(s) placed on “bottom” as shown in example 610 may project the light in an angle different from the that of the placement where the DOEs are placed on the “side” or are distributed surrounding the imaging device. The DOE may also have various configuration, shapes or sizes.

FIG. 7 shows an example of configuration 700 utilizing multiple fibers. As described above, in some embodiments, multiple optic fibers 701 may be utilized. In some cases, each optic fiber may be a single mode fiber. In the illustrated example, the multiple fibers may each carry a different wavelength. In some cases, one of the optic fibers may be a multimode fiber.

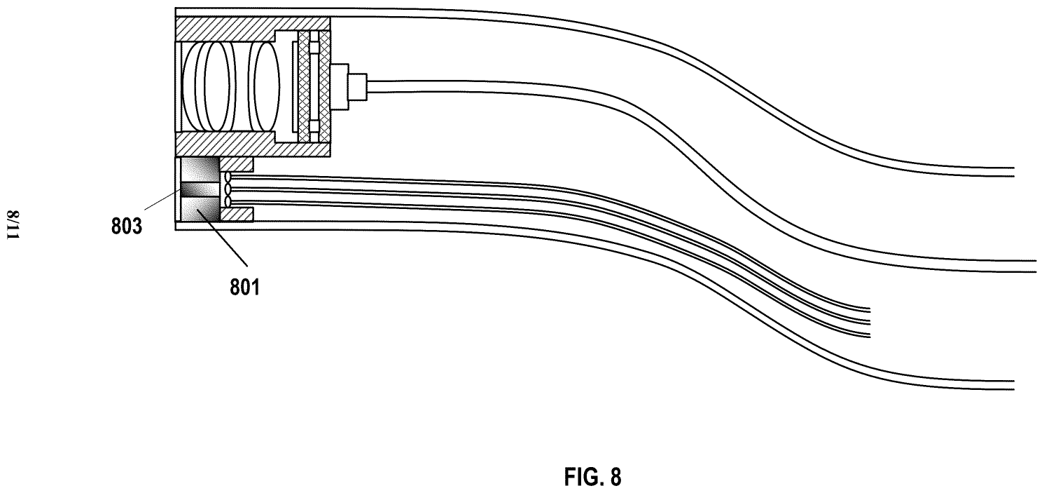

As described above, employing multiple single mode optic fibers may allow for selecting optical elements for different light sources. In the example illustrated in FIG. 7, the multiple fibers may share the same DOE 703. Alternatively, the multiple fibers may each have an individual DOE as shown in FIG. 8. For example, different optical elements such as DOEs 801, 803 may be selected corresponding to the properties of the light coming from each fiber where the light may be different. For example, different optic elements may be selected for LED light, laser beam, different wavelengths (e.g., UV. IR, etc.), different desired diffusion pattern, and the like.

The placement of the DOEs may have various configurations or layouts. For example, the multiple DOEs may be located on one side of the imaging sensor as shown in FIG. 7 or FIG. 8. Alternatively, as shown in FIG. 9, the DOEs 901 may be distributed spatially across the distal tip such as surrounding the imaging sensor 903. In some cases, each optic fiber may correspond to an individual DOE. In some cases, a portion of the optic fibers may share a DOE whereas others may each have an individual DOE.

In some cases, each DOE may be different in a variety of ways depending on the configuration. For instance, the DOE may be matched to the dominant wavelength of light that is coupled to it by its fiber by aligning the operating wavelength DOE to each laser wavelength. Based on the DOE(s) placement, the corresponding spatial response may be different. For example, the DOE(s) placed on “bottom” may project the light in an angle different from the that of the DOEs placed on the “top” or DOEs that are distributed surrounding the imaging device.

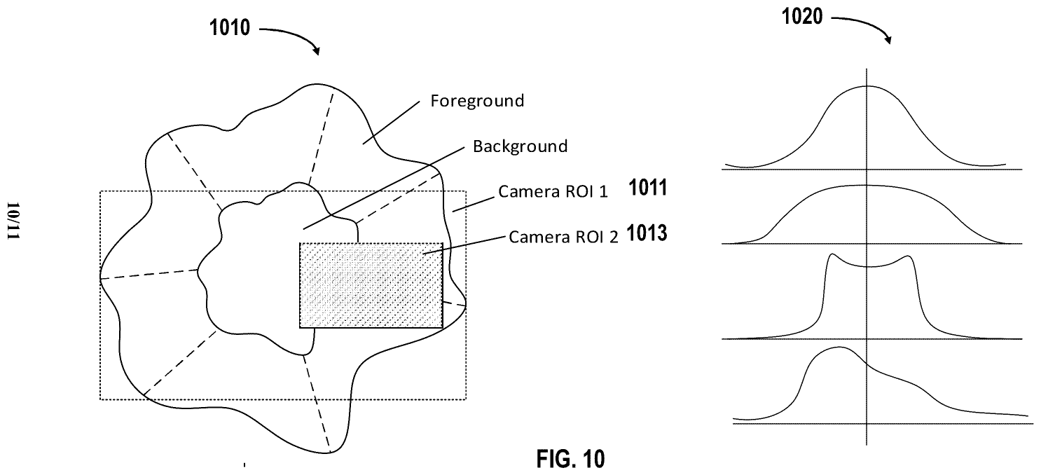

In some cases, multiple sets of DOEs may be utilized where the first set of DOEs may be configured to project the illumination at a first angle such as for illuminating a target field close to the camera, and the second set of DOEs may be configured to project the illumination at a different angle for illuminating a target field further from the camera. This beneficially allows for controlling spatial distribution of illumination or illuminating selected regions of a target scene. FIG. 10 shows an example of controlling the illumination for different regions of a target scene. In the example 1010, the imaging device such as a camera may be directed at different region of interests (ROIs) 1011, 1013. In the first region (ROI 1 1011), the camera may be zoomed out and the illumination may be desired to illuminate the entirety of a tissue such as a long cylindrical anatomical structure. In the second region (ROI 2 1013), the camera may be closer to the tissue and may capture image of a relatively flat scene that requires a different illumination. The illumination requirements of these two regions may be very different that a first set of DOEs may be configured to illuminate the first region at a distant further from the camera and a second set of DOEs may be configured to illuminate the second region with a different illumination pattern suitable for flat scene or near field illumination. In some cases, the DOE may also shape the illumination or modify the intensity distribution profile as shown in the examples 1020. This may beneficially allow for flexibility in optimizing the illumination for different applications. For example, during a procedure where the imaging device captures images in tunnels of a certain size, the DOEs may shape the light or modify the intensity distribution for illuminating the tunnel.

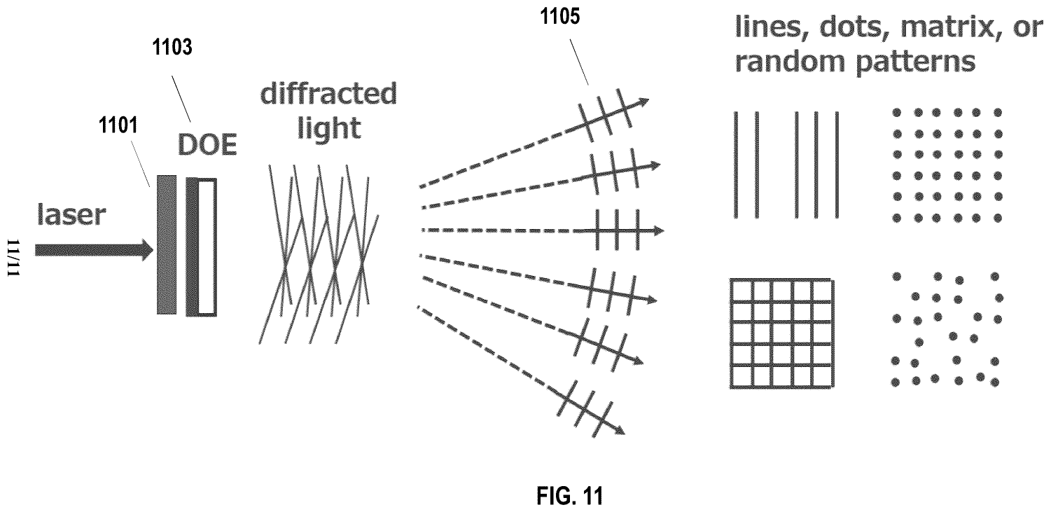

In some cases, additional DOEs with different functions may be employed. FIG. 11 schematically shows an example of using a combination of different DOEs for achieving additional information about a target scene. As shown in FIG. 11 the system may employ a first DOE 1101 for light shaping (e.g., may be constructed holographically or lithographically, or one or more lenses) and a second DOE 1103 for creating structured light (e.g., projected light pattern such as lines, dots, or random pattern 1105) which is utilized for depth mapping or depth sensing. In some cases, the second DOE 1103 may be a diffractive optic used as an active optical approach (e.g., to generate structured light 1105) to extract depth information about a target scene. The first DOE and second DOE may be placed in the same light path corresponding to the same fiber optic. Alternatively, the second DOE may be placed at a separate light path to project structed light into a scene that may or may not the same as the scene illuminated via the first DOE.

While the configuration of the distal tip and the illumination system are described with respect to the endoscope, it is noted that the subject matter of this disclosure is not limited to the example discussed above. For example, the light source may be utilized in a robotic medical system and the catheter may or may not be single-use.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A medical device comprising:
an articulating elongate member comprising a distal end, wherein an imaging sensor is located at the distal end of the articulating elongate member;
one or more laser light sources located at a proximal component coupled to the articulating elongate member, wherein the one or more laser light sources generate light transmitted through one or more optic fibers; and
one or more optical elements located at the distal end of the articulating elongate member, wherein the one or more optical elements receive the light via the one or more optic fibers and are configured to adjust a distribution of the light for illuminating a target scene based at least in part on a property of the imaging sensor or a property of the target scene.

2. The medical device of claim 1, further comprising a coupler to mix the light from the one or more laser light sources into white light.

3. The medical device of claim 1, wherein the articulating elongate member is releasably coupled to the proximal component, and wherein the articulating elongate member is disposable.

4. The medical device of claim 1, further comprising a laser speckle reducer located at the proximal component.

5. The medical device of claim 1, wherein the one or more laser light sources comprise at least a red laser diode, a green laser diode and a blue laser diode.

6. The medical device of claim 5, wherein the one or more optical elements are selected based at least in part on the operating wavelength of the light received via a respective optic fiber.

7. The medical device of claim 1, wherein the property of the imaging sensor comprises at least one of field of view, sensitivity, acquisition parameter and exposure.

8. The medical device of claim 7, wherein the distribution of the light is adjusted to match the field of view of the imaging sensor.

9. The medical device of claim 1, wherein the imaging sensor is embedded in the distal end of the articulating elongate member.

10. The medical device of claim 1, wherein the one or more optical elements comprise at least one diffractive optic element.

11. The medical device of claim 10, wherein the one or more optical elements further comprise an optical element for generating structured light that is used for creating a depth map.

12. An illumination system for a medical device comprising:
one or more laser light sources located at a proximal component of the medical device, wherein the proximal component is coupled to an articulating elongate member of the medical device;
one or more optic fibers for transmitting light generated by the one or more laser light to a distal end of the articulating elongate member; and
one or more optical elements located at the distal end of the articulating elongate member, wherein the one or more optical elements receive the light via the one or more optic fibers and are configured to adjust a distribution of the light for illuminating a target scene based at least in part on a property of an imaging sensor or a property of the target scene.

13. The illumination system of claim 12, further comprising a coupler to mix the light from the one or more laser light sources into white light.

14. The illumination system of claim 12, wherein the articulating elongate member is releasably coupled to the proximal component, and wherein the articulating elongate member is disposable.

15. The illumination system of claim 12, further comprising a laser speckle reducer located at the proximal component.

16. The illumination system of claim 12, wherein the one or more laser light sources comprise at least a red laser diode, a green laser diode and a blue laser diode.

17. The illumination system of claim 16, wherein the one or more optical elements are selected based at least in part on the operating wavelength of the light received via a respective optic fiber.

18. The illumination system of claim 12, wherein the property of the imaging sensor comprises at least one of field of view, sensitivity, acquisition parameter and exposure.

19. The illumination system of claim 18, wherein the distribution of the light is adjusted to match the field of view of the imaging sensor.

20. The illumination system of claim 12, wherein the imaging sensor is embedded in the distal end of the articulating elongate member.

21. The illumination system of claim 12, wherein the one or more optical elements comprise at least one diffractive optic element.

22. The illumination system of claim 21, wherein the one or more optical elements further comprise an optical element for generating structured light that is used for creating a depth map.
